(19) United States
(12) Patent Application Publication
(10) Pub. No.: US 2016/0060145 A1
(43) Pub. Date: Mar. 3, 2016

(54) CHELATING AGENT, AND SCALE INHIBITOR, DETERGENT AND WATER TREATMENT METHOD

(71) Applicant: Kabushiki Kaisha Toshiba, Minato-ku (JP)

(72) Inventors: Akiko SUZUKI, Minato (JP); Tomohito IDE, Inagi (JP); Taro FUKAYA, Setagaya (JP); Toshihiro IMADA, Kawasaki (JP)

(73) Assignee: Kabushiki Kaisha Toshiba, Minato-ku (JP)

(21) Appl. No.: 14/675,997

(22) Filed: Apr. 1, 2015

(30) Foreign Application Priority Data
Sep. 3, 2014 (JP) 2014-179553

(51) Int. Cl. C02F 1/52; C02F 1/44; C11D 3/37; C08K 5/20; C07C 235/12; C11D 3/00

(52) U.S. Cl. CPC C02F 1/5272; C07C 235/12; C11D 3/0036; C11D 3/3723; C08K 5/20; C02F 1/44; C02F 2101/20

(57) ABSTRACT

A chelating agent of the embodiment is an amide compound in which a hydrogen atom of an —NH— site or an —NH₂— site in an amine compound is substituted with a functional group derived from diglycolic acid. The amine compound is selected from the group consisting of a compound represented by a following general formula (1) and a polyethyleneimine having a molecular weight of 200 to 100,000. In the general formula (1), x, y and z respectively represent a number of repetitions for —CH₂—CH₂—NH—, x represents an integer of 1 to 10, y represents an integer of 0 to 5, and z represents an integer of 0 to 5.

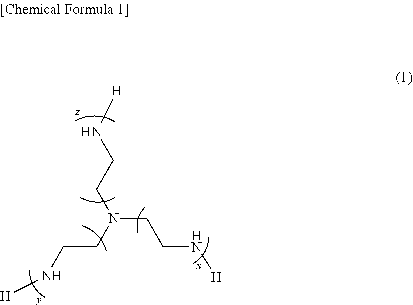

### CROSS-REFERENCE TO RELATED APPLICATION

This application is based upon and claims the benefit of priority from Japanese Patent Application No. 2014-179553, filed Sep. 3, 2014, the entire contents of which are incorporated herein by reference.

### FIELD

Embodiments of the present invention relate to a chelating agent, and a scale inhibitor, a detergent and a water treatment method.

### BACKGROUND

Tap water contains minerals such as calcium.

For example, mineral component-containing deposited materials (scale) can be found in pipes of a water-heating apparatus, and this scale tends to cause clogging of pipes. The scale mainly contains calcium carbonate which is precipitated in water.

Detergents can be affected by mineral components such that bubbling is deteriorated and detergency is decreased.

In contrast, it is conventional to use a chelating agent which coordinates to a mineral component so as to form a chelate compound and has the effect of separating mineral components from water.

A chelating agent is used also for removal and recovery of metal ions contained in water to be treated such as a chemical solution or waste water.

However, the chelating effect may not be sufficiently obtained depending on liquid property of water to be treated. For example, when water to be treated is strongly acidic, it is impossible to sufficiently obtain the chelating effect.

### BRIEF DESCRIPTION OF DRAWINGS

FIG. 1 is a schematic diagram illustrating a treatment system for explaining a water treatment method of a first embodiment.

FIG. 2 is a schematic diagram illustrating a membrane separation apparatus for explaining a water treatment method of a second embodiment.

### DETAILED DESCRIPTION

A chelating agent of the embodiment is an amide compound in which a hydrogen atom of an —NH— site or an —NH₂— site in an amine compound is substituted with a functional group derived from diglycolic acid. The amine compound is selected from the group consisting of a compound represented by a following general formula (1) and a polyethyleneimine having a molecular weight of 200 to 100,000.

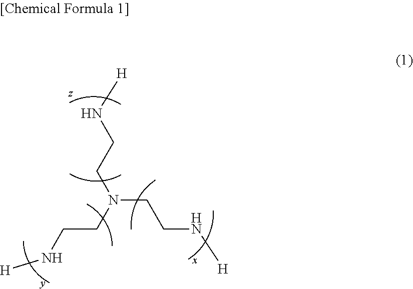

(In the formula, x, y and z respectively represent a number of repetitions for —CH₂—CH₂—NH—, x represents an integer of 1 to 10, y represents an integer of 0 to 5, and z represents an integer of 0 to 5.)

Hereinafter, the chelating agent of an embodiment is described.

The chelating agent of the present embodiment is an amide compound.

The amide compound refers to a compound having at least one structure selected from the group consisting of the —NH—C(═O)— structure and the >N—C(═O)— structure. The amide compound constituting the chelating agent of the present embodiment can have a carboxylic group, and this carboxylic group can form a carboxylate salt.

The amide compound is produced by substituting a hydrogen atom of an —NH— site or an —NH₂— site in an amine compound with a functional group derived from diglycolic acid.

Both of the two hydrogen atoms of an —NH₂— site in an amine compound can be substituted with the functional groups, and either one of the two hydrogen atoms can be substituted with the functional group.

However, it is not required that the hydrogen atoms of all of the —NH— sites and —NH₂— sites in an amine compound are substituted with the functional groups.

Hereinafter, the amine compound is described.

The amine compound is selected from the group consisting of a compound represented by a following general formula (1) and a polyethyleneimine having a molecular weight of 200 to 100,000.

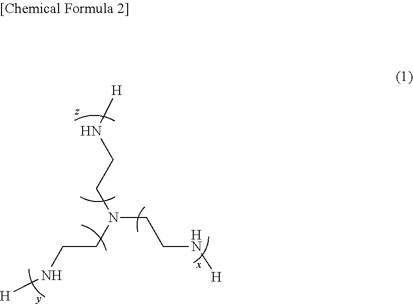

(In the formula, x, y and z respectively represent a number of repetitions for —CH₂—CH₂—NH—, x represents an integer of 1 to 10, y represents an integer of 0 to 5, and z represents an integer of 0 to 5.)

In the general formula (1), x represents an integer of 1 to 10, and preferably an integer 1 to 5.

Also, y represents an integer of 0 to 5, and preferably 0 or 1.

Also, z represents an integer of 0 to 5, and preferably 0 or 1.

Preferable examples of the compound represented by the general formula (1) include ethylenediamine, diethylenetriamine, tris(2-aminoethyl)amine, triethylenetetramine, tetraethylenepentamine and pentaethylenehexamine.

The molecular weight of the polyethyleneimine is within a range of 200 to 100,000, preferably within a range of 300 to 10,000, and more preferably within a range of 400 to 3,000.

When the molecular weight of the polyethyleneimine is not lower than the aforementioned lower limit, it is likely to exert the chelating effect. On the other hand, when the molecular weight of the polyethyleneimine is not higher than the aforementioned upper limit, the solubility is increased. Also, as the molecular weight of the polyethyleneimine is increased within the aforementioned range, the effect of separating heavy metals from heavy metal-containing water to be treated is increased.

Herein, the molecular weight of the polyethyleneimine refers to a number average molecular weight which is a value measured by an ebullioscopic method.

Hereinafter, the functional group derived from diglycolic acid is described.

Examples of the functional group include the group represented by the following chemical formula (2) and the group represented by the following general formula (3).

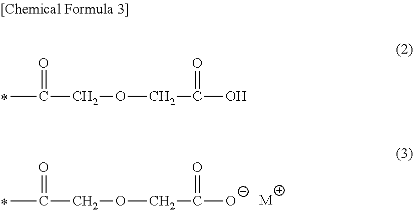

(In the general formula (3), M⁺ represents a counter cation. In the formulas, the symbol of “*” represents a chemical bond.)

In the general formula (3), examples of M⁺ include alkali metal cations such as a sodium ion and a potassium ion; an ammonium ion; and an alkanolammonium ion such as a monoethanolammonium ion, a diethanolammonium ion and a triethanolammonium ion. Of these, alkali metal cations are preferred because the solubility becomes good.

In the formulas (2) and (3), a chemical bond represented by the symbol of “*” is connected to a nitrogen atom instead of a hydrogen atom of an —NH— site or an —NH₂— site in the amine compound.

In the group represented by the formulas (2) and (3), it is speculated that 3 sites, i.e. two oxygen atoms of the carbonyl groups (>C(═O)) and one oxygen atom of the ether bond (—O—), be respectively coordinately bonded to a metal ion (a central atom of a complex).

Examples of the chelating agent of the present embodiment include the amide compounds, which are respectively represented by the following chemical formulas (1-1) to (1-6), and the polymers in which a hydrogen atom of an —NH— site or an —NH₂— site in a polyethyleneimine having a molecular weight of 200 to 100,000 is substituted with the aforementioned functional group R.

The number of the functional group R per molecule in the polyethyleneimine is preferably within a range of 2 to 1,000 and more preferably within a range of 2 to 100. When the number of the functional group R is not lower than the aforementioned preferable lower limit, it is likely to obtain the chelating effect. On the other hand, when the number of the functional group R is higher than the aforementioned preferable upper limit, the solubility may be decreased.

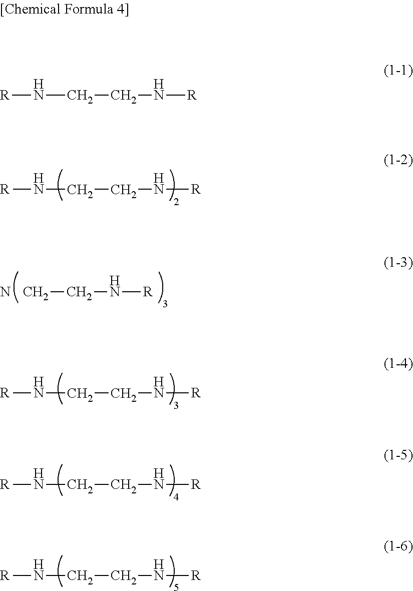

(In the chemical formulas, R represents the group represented by the chemical formula (2) or the group represented by the general formula (3). A plurality of R existing in one molecule can be different or the same.)

Hereinafter, the production method of the chelating agent of the present embodiment is described.

The chelating agent of the present embodiment includes a carboxylic acid-typed amide compound and a carboxylate salt-typed amide compound. For example, a carboxylic acid-typed amide compound is produced by mixing the amine compound (the compound represented by the general formula (1) or the polyethyleneimine having the predetermined molecular weight) and the diglycolic anhydride in a solvent.

Also, a carboxylate salt-typed amide compound is produced by mixing the amine compound and the diglycolic anhydride in a solvent, followed by adding a base therein and mixing.

The mixing ratio of the amine compound and the diglycolic anhydride is appropriately determined in consideration of the number of an —NH— site or an —NH₂— site contained in the amine compound, for example.

The temperature condition for mixing of the amine compound and the diglycolic anhydride is preferably 50° C. or lower and more preferably within a range of 5° C. to 30° C.

The mixing time for mixing of the amine compound and the diglycolic anhydride is appropriately determined in consideration of a temperature condition and a blending amounts, etc. For example, the mixing time is preferably within a range of 0.2 to 24 hours and more preferably within a range of 1 to 6 hours.

Examples of the solvent include methylene chloride, chloroform, methanol, ethanol, propanol, isopropanol, acetone, ethyl acetate, acetonitrile, toluene, benzene, water and 2-methoxyethanol.

Examples of the base include alkali metal hydroxides such as sodium hydroxide and potassium hydroxide; ammonia; and alkanolamines such as monoethanolamine, diethanolamine and triethanolamine.

The obtained carboxylic acid-typed amide compound or carboxylate salt-typed amide compound can be directly used as the chelating agent after evaporating a solvent therein. Alternatively, the carboxylic acid-typed amide compound or carboxylate salt-typed amide compound subjected to the purification using column chromatography, etc. can be used as the chelating agent.

Because the chelating agent of the embodiment is the amide compound in which a hydrogen atom of an —NH— site or an —NH₂— site in the specific amine compound is substituted with a functional group derived from the diglycolic acid, it is possible to exert the high chelating effect over a wider pH range.

The chelating agent of the embodiment shows the high chelating effect even under a strongly acidic condition, and also, the solubility is good.

Also, the chelating agent of the embodiment shows the high chelating effect on both of heavy metals and light metals.

The chelating agent of the embodiment is preferably used in applications such as a detergent, an inorganic pigment dispersant, a fiber-treating agent, a water-treating agent, an electrolytic peeling agent and a bleaching assistant for wood pulp.

Hereinafter, the scale inhibitor of the embodiment is described.

The scale inhibitor of the present embodiment includes the chelating agent of the embodiment. The chelating agent can be used alone or in combination of two or more.

In the scale inhibitor, the content of the chelating agent of the embodiment is preferably 0.1 mass % or higher with respect to the total amount of the scale inhibitor, and more preferable within a range of 50 to 100 mass %.

The scale inhibitor can include any component other than the chelating agent. Examples of the component other than the chelating agent include a corrosion inhibitor and a preservative.

The used amount of the scale inhibitor of the present embodiment is appropriately determined in consideration of the amount of an ion contained in water, etc. For example, when the scale inhibitor is added in the water to be treated dissolving calcium carbonate 40 ppm (by weight) therein, the amount of the scale inhibitor is preferably within a range of 40 to 120 mg/L with respect to the water to be treated (L), and more preferably within a range of 50 to 60 mg/L. Herein, the aforementioned amount of the scale inhibitor is the reduced value of the chelating agent amount (mg). When the reduced value of the chelating agent amount is not lower than the preferable lower limit, it is more likely to suppress the precipitation of calcium carbonate. On the other hand, even when the reduced value of the chelating agent amount exceeds the preferable upper limit, the chelating effect is not enhanced.

Because the scale inhibitor of the embodiment includes the chelating agent of the embodiment, it is possible to suppress the precipitation of calcium carbonate, etc. in water to be treated and to prevent the scale from attaching to pipes, etc.

Hereinafter, the detergent of the embodiment is described.

The detergent of the present embodiment includes the chelating agent of the embodiment. The chelating agent can be used alone or in combination of two or more.

The applications of the detergent of the embodiment are not particularly limited, and the detergent is preferred for clothing or for a member having a hard surface such as tableware or bathroom.

The form of the detergent can be a solid form (such as a granular form) or a liquid form, and these detergents can be produced by conventionally known production methods.

The detergent is blended with a surfactant which is a general cleaning component as a component other than the chelating agent.

Examples of a surfactant include anionic surfactants such as an alkylbenzene sulfonate, an alkyl sulfate, a polyoxyethylene alkyl ether sulfate and an olefin sulfonate salt; and a nonionic surfactant such as a polyoxyethylene alkyl ether.

In the detergent, the content of a surfactant is preferably within a range of 5 to 40 mass % with respect to the total amount of the detergent.

In the detergent, the content of the chelating agent of the embodiment is preferably 0.01 to 10 mass % with respect to the total amount of the detergent, and more preferable within a range of 0.1 to 1 mass %. When the content of the chelating agent is not lower than the preferable lower limit, it is more likely to maintain the foaming property of a surfactant. On the other hand, even when the content of the chelating agent exceeds the preferable upper limit, the chelating effect is not enhanced.

The detergent of the present embodiment can optionally include any component, which is other than the chelating agent and the surfactant, such as a builder, an enzyme, a fragrance, a foam inhibitor or a fluorescent bleaching agent.

Examples of a builder include inorganic builders such as a soda ash, a sulfate salt, a silicate salt, a zeolite and a tripoly phosphate salt; and organic builders such as a polyacrylate salt, a polymaleate salt, a citrate salt, an ethylenediamine tetraacetate salt and a nitrilotriacetate salt.

Because the detergent of the embodiment includes the chelating agent of the embodiment, it is possible to suppress the deterioration of the foaming property and to exert the good cleaning effect.

Hereinafter, the water treatment method of the embodiment is described.

### (Water Treatment Method of First Embodiment)

The water treatment method of the first embodiment is the method including: a mixing step in which a heavy metal-containing water to be treated and the chelating agent of the embodiment are mixed so as to obtain a mixed solution; and a precipitation step in which the mixed solution and a precipitant are mixed so as to produce heavy metal-containing precipitate and to obtain treated water in which the heavy metal has been removed from the water to be treated.

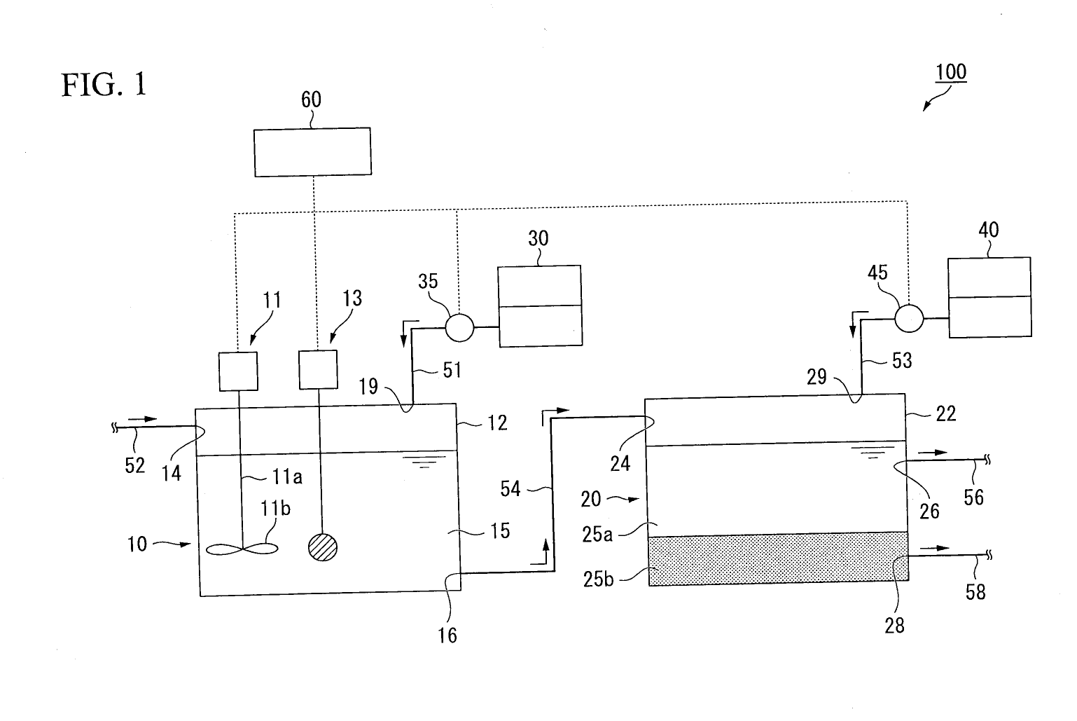

Hereinafter, the water treatment method of the first embodiment is described with reference to FIG. 1.

The water treatment method of the first embodiment can be carried out using the water treatment system 100 illustrated in FIG. 1, for example.

The water treatment system 100 illustrated in FIG. 1 includes the mixing apparatus 10 and the solid-liquid separation apparatus 20. The overall water treatment system 100 is configured to be controlled by the controller 60.

The mixing apparatus 10 includes the mixing tank 12, the stirring apparatus 11 and the pH meter 13.

The mixing tank 12 is configured to form the introduction port 14 for the water to be treated 15, the discharge port 16 for the mixed solution, and the introduction port 19 for the chelating agent, respectively. The introduction port 14 is connected to the supply pipe 52 through which the water to be treated 15 flows. The discharge port 16 is connected to the flow path 54 through which the mixed solution flows. The introduction port 19 is connected to the chelating agent-storing tank 30 through the supply pipe 51. The supply pipe 51 is equipped with the pump 35 between the chelating agent-storing tank 30 and the introduction port 19.

The stirring apparatus 11 includes the stirring shaft 11a and the stirring blades 11b provided at the end of the stirring shaft 11a.

In FIG. 1, the water to be treated 15 is supplied to the mixing tank 12.

The solid-liquid separation apparatus 20 includes the apparatus body 22. The apparatus body 22 is configured to form the introduction port 24 for the mixed solution, the discharge port 26 for the treated water 25a, the discharge port 28 for the precipitate 25b, and the introduction port 29 for the precipitant, respectively.

The introduction port 24 is connected to the flow path 54 through which the mixed solution flows. The discharge port 16 and the introduction port 24 are connected through the flow path 54. The discharge port 26 is connected to the outflow pipe 56 through which the treated water 25a flows. The discharge port 28 is connected to the discharge pipe 58 through which the precipitate 25b flows. The introduction port 29 is connected to the precipitant-storing tank 40 through the supply pipe 53 for the precipitants. The supply pipe 53 is equipped with the pump 45 between the precipitant-charging tank 40 and the introduction port 29.

In FIG. 1, the mixed solution is supplied to the apparatus body 22. The mixed solution is separated into the supernatant liquid (the treated water 25a) and the precipitate 25b.

Hereinafter, the mixing step is described.

In the mixing step, the heavy metal-containing water to be treated and the chelating agent of the embodiment are mixed so as to obtain the mixed solution. The mixing of the both forms the heavy metal-containing chelate compound. The chelate compound is dissolved in the water to be treated.

The mixing step is carried out, for example, in the following manner by using the mixing apparatus 10.

In the mixing apparatus 10, the water to be treated 15 is flowed through the supply pipe 52, and is supplied from the introduction port 14 to the mixing tank 12. Subsequently, the pH of the water to be treated 15 stored in the mixing tank 12 is adjusted by the pH meter 13 according to the type of heavy metal to be separated, etc. After the pH adjustment, the water to be treated 15 is stirred by the stirring apparatus 11. During the stirring, the pump 35 is started to thereby feed the chelating agent from the chelating agent-storing tank 30 to the mixing tank 12. Then, in the mixing tank 12, the water to be treated 15 and the chelating agent are mixed so as to prepare the mixed solution.

The heavy metals contained in the water to be treated is the metal having a density of 4.0 g/cm³ or higher. Examples of the heavy metal include iron, cobalt, copper, gallium, dysprosium, neodymium, chromium, manganese, nickel, lead, zinc and cadmium.

In the water to be treated, the concentration of the heavy water is not particularly limited, and for example, the concentration of the heavy water is preferably within a range of about 0.1 to 1,000 mg/L with respect to the total amount of the water to be treated. The water to be treated can contain the two or more heavy metals.

The pH of the water to be treated can be appropriately adjusted in consideration of the type of heavy metal, etc. The pH of the water to be treated can be adjusted to be strongly acidic. The pH of the water to be treated, which shows strong acidity, is, for example, 2 or lower and more specifically 1.5 or lower.

The pH adjustment of the water to be treated is carried out using an acid such as hydrochloric acid or sulfuric acid; and a base such as sodium hydroxide.

Regarding the mixing ratio of the water to be treated and the chelating agent, the amount of the chelating agent is preferably within a range of 5 to 1,000 mg with respect to the water to be treated 100 mL, and more preferably within a range of 10 to 100 mg.

When the proportion of the chelating agent is not lower than the preferable lower limit, it is possible to obtain the sufficient effect to separate the heavy metals from the water to be treated. On the other hand, when the proportion of the chelating agent is not higher than the preferable upper limit, it is possible to well suppress the unmelted residue of the chelating agent.

Herein, the pH adjustment of the water to be treated is carried out in the mixing step described above, but it is not necessarily to carry out the pH adjustment of the water to be treated depending on the type of heavy metal to be separated. As described above, the pH adjustment of the water to be treated enhances the selective separation of the predetermined heavy metals from the water to be treated.

Hereinafter, the precipitation step is described.

In the precipitation step, the precipitant and the mixed solution obtained in the mixing step are mixed so as to produce the heavy metal-containing precipitate. Then, the precipitate is removed from the mixed solution to thereby obtain the treated water in which the heavy metals have been removed from the water to be treated.

The precipitation step is carried out, for example, in the following manner by using the solid-liquid separation apparatus 20.

In the solid-liquid separation apparatus 20, the mixed solution obtained in the mixing step is flowed through the flow path 54, and is supplied from the introduction port 24 to the apparatus body 22. Subsequently, the pump 45 is started to thereby feed the precipitant from the precipitant-storing tank 40 to the apparatus body 22. Then, the mixed solution and the precipitant are mixed in the apparatus body 22 so as to form the precipitate 25b, and the chelate compound dissolved in the mixed solution is separated from the mixed solution. After the precipitation, the supernatant liquid is discharged as the treated water 25a from the discharge port 26 to the outside of the apparatus body 22. The discharged treated water 25a is fed through the outflow pipe 56 to the next treatment apparatus (unillustrated). The precipitate 25b is discharged from the discharge port 28 to the outside of the apparatus body 22. The discharged precipitate 25b is fed through the outflow pipe 58 to the next treatment apparatus (unillustrated).

The precipitant can be appropriately selected in consideration of the type of heavy metal, etc., and can be used alone or in combination of two or more. Of these, at least one selected from the group consisting of silica, alumina, talc, silica gel and zeolite is preferred as the precipitant because the precipitation in the mixed solution is more likely to occur.

Regarding the mixing ratio of the mixed solution and the precipitant, the amount of the precipitant is preferably within a range of 10 to 1,000 mg with respect to the mixed solution 100 mL, and more preferably within a range of 100 to 500 mg.

When the proportion of the precipitant is not lower than the preferable lower limit, the precipitation in the mixed solution is more likely to occur. On the other hand, when the proportion of the precipitant is not higher than the preferable upper limit, the production amount of the precipitate is suppressed to an appropriate amount.

According to the water treatment method of the first embodiment, it is possible to separate and remove the heavy metals from the heavy metal-containing water to be treated.

Moreover, in the water treatment method of the first embodiment, the chelating agent is well dissolved even in the water to be treated which shows strong acidity. The chelating agent of the embodiment can coordinate to the heavy metal even under a strongly acidic condition so as to form the chelate compound. Thus, in the water treatment method of the first embodiment, it is possible to obtain the high chelating effect. Then, when the precipitant is added, the chelate compound and the precipitant are interacted to thereby form the heavy metal-containing precipitate. For this reason, it is possible to obtain the treated water in which the heavy metals have been removed from the water to be treated.

Particularly, the water treatment method of the first embodiment is preferable to remove rare earth metals among the heavy metals from the water to be treated.

### (Water Treatment Method of Second Embodiment)

The water treatment method of the second embodiment is the method including: a mixing step in which a heavy metal-containing water to be treated and the chelating agent of the embodiment are mixed so as to obtain a mixed solution; and a membrane separation step in which the mixed solution is subjected to a membrane separation treatment so as to obtain treated water in which the heavy metal has been removed from the water to be treated.

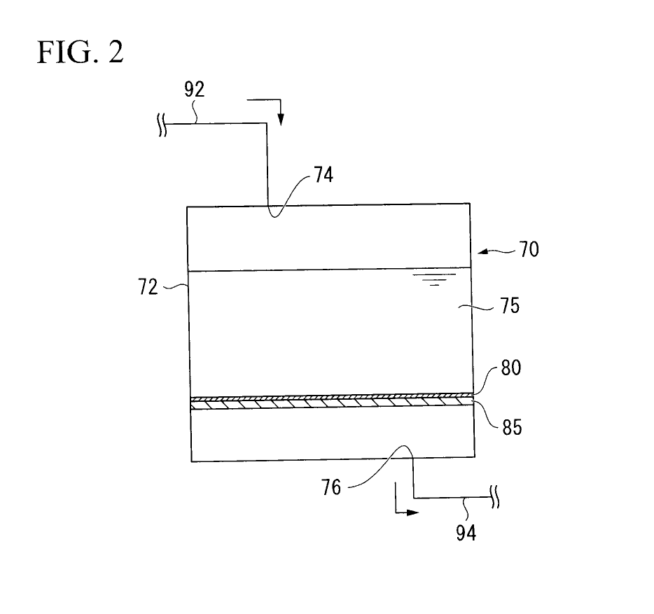

The water treatment method of the second embodiment can be carried out using the water treatment system obtained by changing the solid-liquid separation apparatus 20 in the water treatment system 100 illustrated in FIG. 1 into the membrane separation apparatus 70 illustrated in FIG. 2, for example.

The mixing step in the second embodiment is the same as the mixing step in the aforementioned first embodiment.

Hereinafter, the membrane separation step is described.

In the membrane separation step, the mixed solution is subjected to the membrane separation treatment so as to obtain treated water in which the heavy metal has removed from the water to be treated.

The membrane separation step is carried out, for example, in the following manner by using the membrane separation apparatus 70.

The membrane separation apparatus 70 includes the apparatus body 72. The apparatus body 72 is configured to form the introduction port 74 for the mixed solution 75 and the drainage port 76 for the treated water. The introduction port 74 is connected to the inflow pipe 92, and the drainage port 76 is connected to the outflow pipe 94. The separation membrane 80 is installed in the inside of the apparatus body 72. The inside of the apparatus body 72 is divided by the separation membrane 80 into the space in which the introduction port 74 is opened and the space in which the drainage port 76 is opened. The separation membrane 80 is supported by the supporting membrane 85.

In FIG. 2, the mixed solution 75 is supplied to the apparatus body 72.

Examples of the separation membrane 80 include a nanofiltration membrane (such as the product name NTR-7450HG manufactured by Nitto Denko Corporation), a reverse osmosis membrane (such as the product name NTR-7250HG manufactured by Nitto Denko Corporation), an ultrafiltration membrane (such as the product name NTU-2120 manufactured by Nitto Denko Corporation), and these are appropriately used according to the molecular size of the chelating agent.

In the membrane separation step, the mixed solution 75 obtained in the mixing step is flowed through the inflow path 92, and is supplied from the introduction port 74 to the apparatus body 72. The mixed solution 75 supplied to the apparatus body 72 is stored in the space in which the introduction port 72 is opened.

During the membrane separation treatment, the pressure inside the apparatus body 72 is preferably within a range of about 0.1 to 3 MPa and more preferably within a range of about 0.5 to 1 MPa. By adjusting the pressure inside the apparatus body 72 within the preferable range, the mixed solution 75 permeates the separation membrane 80 more rapidly. When the mixed solution 75 permeates the separation membrane 80, the chelate compound dissolved in the mixed solution 75 is separated by the separation membrane 80 from the mixed solution 75.

The liquid which permeated the separation membrane 80 is discharged as the treated water from the discharge port 76 to the outside of the apparatus body 72. The discharged treated water is fed through the outflow pipe 94 to the next treatment apparatus (unillustrated).

According to the water treatment method of the second embodiment, it is possible to separate and remove the heavy metals from the heavy metal-containing water to be treated.

Moreover, in the water treatment method of the second embodiment, the chelating agent is well dissolved even in the water to be treated which shows strong acidity, and the high chelating effect is shown. The pH of the water to be treated, which shows strong acidity, is, for example, 2 or lower, more specifically 1.5 or lower, and particularly 1.0 or lower. Even under such a strongly acidic condition, the chelating agent of the embodiment coordinates to the heavy metal so as to form the chelate compound. Then, when the membrane separation treatment is carried out, the chelate compound cannot permeate the separation membrane, and thus is separated from the water to be treated. For this reason, it is possible to obtain the treated water in which the heavy metals have removed from the water to be treated.

According to at least one of the embodiments described above, because the chelating agent is the amide compound in which a hydrogen atom of an —NH— site or an —NH₂— site in the specific amine compound is substituted with a functional group derived from the diglycolic acid, it is possible to exert the high chelating effect over a wider pH range.

Moreover, in at least one of the embodiments described above, the high chelating effect is shown even under a strongly acidic condition.

### EXAMPLES

Hereinafter, the chelating agents used in the examples are described.

As the amine compound, ethylenediamine, diethylenetriamine, tris(2-aminoethyl)amine, triethylenetetramine, tetraethylenepentamine, pentaethylenehexamine, polyethyleneimine having the molecular weight of 600 and polyethylene imine having the molecular weight of 1,800 are respectively used.

### Examples 1-4 and 6-9

Diglycolic anhydride and the amine compound corresponding to the each Example are mixed in chloroform of 25° C. for 0.5 hours.

Then, the chloroform is evaporated to thereby obtain the product (the amide compound). The resulting product is used directly as the chelating agent.

The aforementioned procedure produces the respective chelating agents of Examples 1-4 and 6-9 which are the amide compound in which a hydrogen atom of an —NH— site or an —NH₂— site in the amine compound is substituted with the group represented by the chemical formula (2).

Herein, the chelating agents of Examples 1-4, 6 and 7 are the compounds represented by the chemical formulas (1-1) to (1-6) (R in the respective formulas represents the group represented by the chemical formula (2)), respectively.

The chelating agent of Example 8 is the polymer compound in which a hydrogen atom of an —NH— site or an —NH₂— site in the polyethyleneimine having a molecular weight of 1,800 is substituted with the group represented by the chemical formula (2) (the number of the functional group R per molecule is within a range of 5 to 15) (PEI(1800)-R).

The chelating agent of Example 9 is the polymer compound in which a hydrogen atom of an —NH— site or an —NH₂— site in the polyethyleneimine having a molecular weight of 600 is substituted with the group represented by the chemical formula (2) (the number of the functional group R per molecule is within a range of 2 to 8) (PEI(600)-R).

### Example 5

Triethylenetetramine and diglycolic anhydride are mixed in chloroform of 25° C. for 0.5 hours. Subsequently, sodium hydride is added therein followed by mixing.

Then, the chloroform is evaporated to thereby obtain the product (the amide compound). The resulting product is used directly as the chelating agent.

The aforementioned procedure produces the chelating agent which is the amide compound in which a hydrogen atom of an —NH— site or an —NH₂— site in the amine compound is substituted with the group represented by the general formula (3) (M⁺ represented the sodium ion).

Herein, the chelating agent of Example 5 is the compound represented by the chemical formula (1-4) (R in the formula represents the group represented by the general formula (3)).

Hereinafter, the scale inhibitor is described.

### Examples 10-18, Comparative Examples 1-2, and Reference Example 1

As the scale inhibitors of Examples 10-18, the respective chelating agents of Examples 1-9 are used.

As the scale inhibitor of Comparative Example 1, trisodium citrate is used.

As the scale inhibitor of Comparative Example 2, triethylenetetramine is used.

The NaHCO₃ aqueous solution having the concentration of 36 mM and the CaCl₂ aqueous solution having the concentration of 18 mM are respectively prepared.

To the glass container, the CaCl₂ aqueous solution 25 mL is added, and the each scale inhibitor 100 mg of the Examples is added and stirred therein.

Then, the NaHCO₃ aqueous solution 25 mL is added therein followed by stirring for 5 hours at room temperature (25° C.), and then the precipitation condition of the scale (the solution was clouded or not) is observed. The results are shown in Table 1. Herein, Reference Example 1 shows the case where the scale inhibitor (the chelating agent) is not added.

TABLE 1

| | | Precipitation of |
|---|---|---|
| | | Scale (solution was |
| Scale Inhibitor | Chelating Agent | clouded or not) |
| Example 10 | (1-1) | Transparent |
| Example 11 | (1-2) | Transparent |
| Example 12 | (1-3) | Transparent |
| Example 13 | (1-4) | Transparent |
| Example 14 | (1-4) | Transparent |
| Example 15 | (1-5) | Transparent |
| Example 16 | (1-6) | Transparent |
| Example 17 | PEI(1800)-R | Transparent |
| Example 18 | PEI(600)-R | Transparent |
| Comparative Example 1 | Trisodium Citrate | Cloudy |
| Comparative Example 2 | Triethylenetetramine | Cloudy |
| Reference Example 1 | (not added) | Cloudy |

It is found that the solution is not clouded when the each scale inhibitor of Examples 10-18 is used. It is found that the solution is clouded when the each scale inhibitor of Comparative Examples 1-2 is used.

From the results described above, it can be understood that the use of the chelating agent of the present embodiment suppresses the precipitation of calcium carbonate; in other words, the chelating effect is exerted.

Hereinafter, the detergent is described.

### Examples 19-27, Comparative Examples 3-4, and Reference Example 2

In the detergents of Examples 19-27, the respective chelating agents of Examples 1-9 are blended.

In the scale inhibitor of Comparative Example 3, trisodium citrate is blended.

In the scale inhibitor of Comparative Example 4, triethylenetetramine is blended.

In the tap water 100 mL having water hardness of 300, sodium lauryl sulfate 10 mg and the each chelating agent 10 mg of the Examples are added and mixed, to thereby prepare the detergent in the liquid state.

The each detergent of the Examples is stirred by using the household mixer for 3 minutes, and then the foaming property is observed. The observation results are shown in Table 2. A represents the case where bubbling is good, B represents the case where bubbling is poor, and C represents the case where bubbling does not occur. Herein, Reference Example 2 shows the detergent in which the chelating agent is not blended.

TABLE 2

| Detergent | Chelating Agent | Foaming Property |
|---|---|---|
| Example 19 | (1-1) | A |
| Example 20 | (1-2) | A |
| Example 21 | (1-3) | A |
| Example 22 | (1-4) | A |
| Example 23 | (1-4) | A |
| Example 24 | (1-5) | A |
| Example 25 | (1-6) | A |
| Example 26 | PEI(1800)-R | A |
| Example 27 | PEI(600)-R | A |
| Comparative Example 3 | Trisodium Citrate | B |
| Comparative Example 4 | Triethylenetetramine | C |
| Reference Example 2 | (not added) | C |

It is found that the detergents of Examples 19-27 have good bubbling and the foaming property is good.

It is found that the detergents of Comparative Examples 3-4 have poor bubbling and the foaming property is poor.

The results described above reveal that the use of the chelating agent of the present embodiment maintains the foaming property of a surfactant even in highly hard water.

Hereinafter, the water treatment method of the first embodiment is described.

### Examples 28-29 and Comparative Example 5

The water treatment illustrated in FIG. 1 is carried out in the following manner by using the water treatment system 100.

As the chelating agent, the chelating agent of Example 8 and the commercially available iminodiacetic acid-typed ion exchange resin are respectively used. As the precipitant, alumina is used.

The mixing step:

The water to be treated, which contains copper (Cu), iron (Fe (III)) and dysprosium (Dy) as the heavy metals, is prepared. The total concentration of the heavy metals in the water to be treated is 100 mg/L. To the water to be treated, 1N hydrochloric acid is added so as to adjust the pH of the waters to be treated at 25° C. to 0.9, 1.3 or 1.5.

The water to be treated 100 mL subjected to the pH adjustment and the chelating agent 20.0 mg are mixed so as to obtain the mixed solution (1).

The precipitation step:

The total amount of the mixed solution (1) obtained in the mixing step is mixed with alumina 1 g. When the resultant solution is left to stand, the white precipitate is produced.

The supernatant liquid is used as the treated water. The respective heavy metal concentrations of the water to be treated and the treated water are measured by ICP spectroscopy. The concentration difference and the following equation are used to calculate the removal rate (%) of the heavy metals. The results are shown in Table 3.

Removal Rate (%)=(1−T₁/T₀)×100

T₀: heavy metal concentration of the water to be treated, T₁: heavy metal concentration of the treated water

As the removal rate (%) is high, the precipitation-producing effect, which is caused by the chelating agent and the precipitant, is high, and the heavy metals are well removed from the water to be treated.

TABLE 3

| | Chelating | pH of Water | Removal Rate (%) | | |
|---|---|---|---|---|---|
| | Agent | to be Treated | Cu | Fe(III) | Dy |
| Example 28 | PEI(1800)-R | 0.9 | 2 | 6 | 85 |
| Example 29 | PEI(1800)-R | 1.5 | 23 | 10 | 90 |
| Comparative | Commercially | 1.3 | 60 | 80 | 5 |
| Example 5 | Available Resin | | | | |

It is found that the removal rate of dysprosium is significantly high in the case of using the chelating agent of Example 8 as compared with the case of using the commercially available iminodiacetic acid-typed ion exchange resin.

From the results described above, it can be understood that, according to the water treatment method of the first embodiment, the rare earth metal can be selectively removed from the water to be treated by combining the chelating agent of the present embodiment with the precipitant under a strongly acidic condition.

Hereinafter, the water treatment method of the second embodiment is described.

### Examples 30-32 and Reference Example 3

The water treatment is carried out in the following manner by using the water treatment system obtained by changing the solid-liquid separation apparatus 20 in the water treatment system 100 illustrated in FIG. 1 into the membrane separation apparatus 70 (the test cell) illustrated in FIG. 2.

As the chelating agent, the chelating agent of Example 8 is used.

The mixing step:

The water to be treated, which contains cobalt (Co), gallium (Ga), iron (Fe (III)) and dysprosium (Dy) as the heavy metals, is prepared. The total concentration of the heavy metals in the water to be treated is 200 mg/L. To the water to be treated, 1N hydrochloric acid is added so as to adjust the pH of the waters to be treated at 25° C. to 0.3, 0.4 or 0.9.

The water to be treated 100 mL subjected to the pH adjustment and the chelating agent 15.9 mg of Example 8 are mixed so as to obtain the mixed solution (2).

The membrane separation step:

The test cell having the same configuration as the membrane separation apparatus 70 illustrated in FIG. 2 is used. As the separation membrane, the nanofiltration membrane (the product name NTR-7450HG manufactured by Nitto Denko Corporation), which is a flat membrane, is used.

The total amount of the mixed solution (2) obtained in the mixing step is flowed from the introduction port to the test cell body, and is stored in the space in which the introduction port is opened. The pressure of the inside of the test cell body is set to 1.5 MPa by using a nitrogen cylinder, and the membrane separation treatment is carried out. Then, the treated water 50 mL is sampled.

The respective heavy metal concentrations of the water to be treated and the treated water are measured by inductively coupled plasma (ICP) spectroscopy. The concentration difference and the following equation are used to calculate the membrane permeability (%) of the heavy metals. The results are shown in Table 4. Herein, Reference Example 3 shows the case where the water to be treated subjected to the pH adjustment and the chelating agent are not mixed and the water to be treated is directly used.

Membrane Permeability (%)=T₃/T₂×100

T₂: heavy metal concentration of the water to be treated, T₃: heavy metal concentration of the treated water

As the membrane permeability rate (%) is low, the chelating effect, which is caused by the chelating agent, is high, and the heavy metals are well removed from the water to be treated.

TABLE 4

| | | pH of Water | | | | |
|---|---|---|---|---|---|---|
| | Chelating | to Be | Membrane Permeability (%) | | | |
| | Agent | Treated | Co | Ga | Fe(III) | Dy |
| Example 30 | PEI(1800)-R | 0.9 | 9.5 | 7.0 | 6.9 | 7.4 |
| Example 31 | PEI(1800)-R | 0.4 | 6.3 | 3.5 | 3.5 | 4.2 |
| Example 32 | PEI(1800)-R | 0.3 | 10.7 | 6.7 | 6.8 | 7.7 |
| Reference | No Mixing | 0.9 | 86.6 | 73.2 | 74.3 | 80.0 |
| Example 3 | | | | | | |

It is found that the membrane permeability of the heavy metals is significantly lowered in the case of using the chelating agent of Example 8. Therefore, it can be understood that the chelating agent of the present embodiment exert the high chelating effect even under a strongly acidic condition.

From the results described above, it can be understood that, according to the water treatment method of the second embodiment, the heavy metals can be well removed from the water to be treated.

While certain embodiments have been described, these embodiments have been presented by way of example only, and are note intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. A chelating agent which is an amide compound in which a hydrogen atom of an —NH— site or an —NH₂— site in an amine compound, which is selected from the group consisting of a compound represented by a following general formula (1) and a polyethyleneimine having a molecular weight of 200 to 100,000, is substituted with a functional group derived from diglycolic acid. 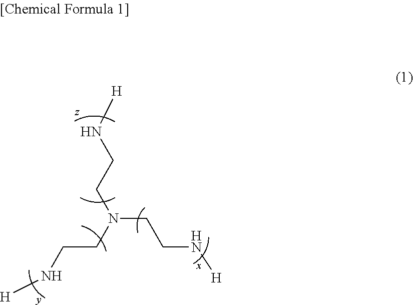
(In the formula, x, y and z respectively represent a number of repetitions for —CH₂—CH₂—NH—, x represents an integer of 1 to 10, y represents an integer of 0 to 5, and z represents an integer of 0 to 5.)

2. A scale inhibitor comprising the chelating agent according to claim 1.

3. A detergent comprising the chelating agent according to claim 1.

4. A water treatment method comprising:
a mixing step in which a heavy metal-containing water to be treated and the chelating agent according to claim 1 are mixed so as to obtain a mixed solution; and
a precipitation step in which the mixed solution and a precipitant are mixed so as to produce heavy metal-containing precipitate and to obtain treated water in which the heavy metal has been removed from the water to be treated.

5. A water treatment method comprising:
a mixing step in which a heavy metal-containing water to be treated and the chelating agent according to claim 1 are mixed so as to obtain a mixed solution; and
a membrane separation step in which the mixed solution is subjected to a membrane separation treatment so as to obtain treated water in which the heavy metal has been removed from the water to be treated.

6. The water treatment method according to claim 4, wherein a pH of the heavy metal-containing water to be treated is 2 or lower.

7. The water treatment method according to claim 5, wherein a pH of the heavy metal-containing water to be treated is 2 or lower.
